(19) United States
(12) Patent Application Publication
(10) Pub. No.: US 2007/0282232 A1
(43) Pub. Date: Dec. 6, 2007

(54) Therapeutic device having auxiliary strap holding positions

(75) Inventor: Keith A. Hoffman, Hudsonville, MI (US)

Correspondence Address: MAYER BROWN LLP, P.O. BOX 2828, CHICAGO, IL, 60690, US

(73) Assignee: BiaCare Corporation

(21) Appl. No.: 11/443,323

(22) Filed: May 30, 2006

(51) Int. Cl. A61F 5/00

(52) U.S. Cl. 602/5

(57) ABSTRACT

A therapeutic device that provides an auxiliary strap holding position for at least one strap where the strap can be temporarily attached to the auxiliary strap holding position on the device to prevent tangling or connection to an unintended surface when the strap is not in a position used to attach the device to a part of a user's body. The auxiliary strap holding position enhances the ease of donning, doffing, and storing the device.

### BACKGROUND OF THE INVENTION

1. Field of the Invention

The present invention relates to therapeutic devices which are applied to a part of the body, such as a limb. More particularly, the invention relates to therapeutic devices having Velcro-type hook-and-loop fasteners for adjustably applying such devices to said body parts.

2. Discussion of the Prior Art

Elastic and inelastic anklets, stockings, wraps and the like have been employed in therapy of the body and limbs. Many designs utilize a means to provide adjustment or aid in the application and removal of a therapeutic device, such as a compression garment, light therapy unit or a thermal treatment wrap.

A popular adjustment means provides Velcro-type hook fasteners, which most often are in a form of what can commonly be referred to as hook fabric, on one or more straps or flaps that can be adjusted and removably attached to a region of loop material, also commonly referred to as loop fabric, on the exterior of the device. It is common to provide sufficiently large regions of hook and loop fabrics, so as to have large surfaces to ensure a secure and firm fixing of the straps in any one of many desired positions.

Due to the nature of the hook and loop fasteners, the straps tend to carry an area having a hook fastener portion, while the device may have at least a portion of its outer surface made with corresponding loop fastener material. This ensures that when in use, the more aggressive hook portion is bound to the outside surface of the device, such as to the outer surface of a garment which wraps around a limb. Indeed, the hook portion on the straps may become firmly fixed to any suitable mating surface with little or no apparent external pressure applied. For example, a dangling strap having hook surfaces accidentally bumping into a loop fabric generally provides sufficient force to firmly embed the hooks into the fabric pile. As a result, most garments using this adjustment means suffer shortcomings in various degrees, particularly in the ease of applying and removing the device without the plurality of straps tangling and inadvertently attaching themselves to various areas of the device or to other fabrics, such as socks or other clothing, during donning, doffing, and storing the device.

For instance, in the prior art, U.S. Pat. No. 3,845,769 relates to a boot having a split sleeve of essentially unyielding material held in position with compression applied by a plurality of bands. The patent illustrates an embodiment in FIGS. 5 and 6 having loop pile on substantially the entirety of exterior of each band, with a region of hook fabric applied on the opposite side proximate the loose end of each band. Since the garment often is self-applied to or removed from a leg while the user is seated in a chair, gravity urges the unattached straps to droop downward, thereby overlapping each other and tangling as the hook portion at the end of each band contacts the outer surface loop portion of the bands in various locations.

Further compounding the donning and adjustment difficulty is the fact that such therapeutic garments frequently are intended to provide gradient pressure to the limb, such that pressure at the distal end of the limb is higher than the pressure applied at the proximal end of the limb. Proper application of the garment often requires the lower straps at the distal end of the limb to be adjusted and removably secured first, followed by sequentially adjusting and removably securing the next strap directly above it. As a result, although the wearer may initially untangle and free the plurality of straps and manage to adjust the first strap, the remaining straps may re-tangle or attach themselves in undesired locations during the process, thereby continuing the wrestling process. For example, the second strap may be fouled by the third, fourth, or fifth strap above it. Even though all of the straps are once again untangled and cleared, the remaining straps may become yet again tangled and fixed to various portions of the garment or the user's clothing.

Yet further adding to the problem is the fact that many persons using such therapeutic devices require the use of therapeutic compression garments because they suffer from edema of the limbs and may have decreased strength. This condition may severely restrict their mobility and ability to reach and manipulate the adjustment straps. As a result, application difficulties that may be perceived as problematic and frustrating by a more able-bodied individual can become an obstacle to receiving proper treatment to those having impaired mobility. For this reason, many patients may be unwilling or unable to cope with the difficulty of donning and adjusting garments of this type and, as a result, may not receive the therapy they require.

Doffing a device of this type provides similar difficulties. As each strap is loosened, preceding straps may tangle and reengage the loop pile of the device in various places, preventing the unobstructed removal of the therapeutic device. Similarly, once the device has been removed, the straps may once again tangle and engage various portions of the loop surfaces, providing a difficult-to-manage tangled mess.

In an alternative embodiment within U.S. Pat. No. 3,845,769, a device shown in FIGS. 1-4 includes bands bent back on themselves to fix the adjustment. While a smaller amount of loop surface presents itself for inadvertent catching of the garment hook portions, the straps in this embodiment still may inadvertently tangle across each other and attach themselves during the donning, doffing, and storage processes. Since the full amount of hook surface provided on each strap may engage a mating loop pile surface, the straps may inadvertently attach themselves with a tenacity that may require exertion to disengage them, nearly equal to that required when they are intentionally attached in their desired position. Moreover, even if the user intentionally folds each strap down prior to donning the garment to prevent tangling, doing so dictates that they must subsequently open each strap using considerable force prior to adjusting and removably fixing each of the plurality of straps, since they likely would be retained with their full connective force.

It should be noted that many therapeutic devices lack the rigidity provided by the essentially unyielding material taught in U.S. Pat. No. 3,845,769. Therapeutic garments having a high degree of flexibility exhibit the same difficulties, but to a greater degree, owing to their propensity to fold and droop during the donning, doffing, and storage processes. In addition, many such garments are constructed such that a large portion, if not substantially the entire exterior surface of the device, comprises a loop fabric type garment.

For example, U.S. Pat. No. 4,215,687 relates to a combination or kit to provide a plurality of body or limb encircling bands comprising an adjustable pressure applying therapeutic device. The plurality of bands have loop fabric or pile on substantially the entirety of the exterior surface with a region of hook fabric applied proximate the ends of each band on the opposite side. While having differences in some aspects of the construction, this device presents the disadvantages previously discussed. In a somewhat similar manner, U.S. Pat. No. 5,120,300 relates to a compression band including male and female connectors and a length of compression band integrally connected to one of the connectors. The connector segment and compression band have loop pile on the entirety of their exterior surfaces with regions of hook fabric applied proximate the ends of each band segment on the opposite side. This type of band device presents very similar disadvantages to those previously discussed.

In yet another example of a prior art device exhibiting similar disadvantages, U.S. Pat. No. 5,254,122 relates to a therapeutic compression device having a slide fastener splicing band and a plurality of parallel compression bands for applying compression to a body or limb. The plurality of bands have loop fabric on substantially the entirety of the exterior surface with a hook fabric portion applied proximate the ends of each band on the opposite side, thus inviting the same problems associated with the aforementioned prior art.

While of a slightly different structure, being cut from one integral piece, the device disclosed in U.S. Pat. No. 5,653,244 relates to a therapeutic compression garment made of flexible, foldable, light weight Velcro-type loop fabric having a central region for wrapping partially around a body part. The device includes a plurality of pairs of bands integrally connected to the central region and extending outwardly in opposite directions. The entire outer surface of the garment comprises a loop fabric or loop portion, with a hook fabric or hook portion applied proximate the ends of each band on the opposite side. This type of structure also presents the disadvantages previously discussed.

U.S. Pat. No. 5,906,206 relates to a therapeutic device adapted for the leg in combination with an ankle-foot wrap for applying therapeutic compression to the leg, ankle and foot. As with the device in U.S. Pat. No. 5,653,244, substantially the entire outer surface of the main garment portion comprises a loop fabric, with a similar attachment region of hook fabric applied proximate the ends of each band on the opposite side, presenting the disadvantages previously discussed. Still further, this can occur with the additional ankle and foot wrap portion having additional hook fastener portions. Quite similar devices are shown in U.S. Pat. No. 5,918,602 and U.S. Pat. No. 6,109,267 which result in the same type of challenges in the donning, doffing and storage processes.

While the loop pile is typically located on the outer surface of therapeutic devices that are to be affixed with straps, at least one known device provides loops on an inner surface. U.S. Pat. No. 3,699,959 relates to a bandage construction comprising an elongated strip of elastic material having a nap or loop fabric on the inner surface, a region of hook fabric positioned to lock the bandage in position after a partial wrapping around the foot and ankle, and a means for releasably locking the end of the bandage after the completion of the wrapping. With this garment, the regions of hook fabric provided are free to inadvertently attach with nearly full connective force to any of the loop fabric of the device or of other clothing articles during donning, doffing, and storage processes.

Hook-and-loop fastener fabrics have been used in other fields involving straps. For example, U.S. Pat. No. 4,168,544 relates to an article-holding system to essentially tie or tether an object to a piece of clothing, and includes a first piece having fabric fastening means thereon and being adapted for attachment to a base member. It further includes a flap having a piece of complementary fabric fastening means on opposite faces at its ends which is attached to the first piece at an intermediate point. With this type of device, the single flap is essentially hooked to the first piece of fabric whether when holding an article or not, thus tending not to encounter the same type of problems. In a similar manner, U.S. Pat. No. 4,236,658 relates to an article-holding system for use on objects to hold and support articles which have a vertical component of weight which is otherwise unsupported. The system includes first and second strips having complementary pieces of fabric fastening material, such as Velcro, thereon. The first strip is attached in a vertical orientation, such as on a jacket. The fabric fastening material of the second strip includes a free-hanging tab which is fastened to the fabric fastening material on the first strip to act as an anti-peel tab. Thus, there is only one strap involved and it is intended for the same fastening position, thereby not presenting the problems encountered with multiple straps becoming inadvertently tangled or misplaced on other attachment surfaces.

This concept of attaching or tethering articles to a main body also is seen in U.S. Pat. No. 7,017,212, which relates to a support pillow. In one embodiment, the support pillow is provided with at least one strap that is attached to the pillow. The strap is configured to attach an article, such as a child's toy, to the pillow. Each such strap is configured to have a fastener system, such as a hook and loop fastener fabric, which allows the strap to be formed into a loop to secure the article. When adjacent strap loops are opened, the straps present a tangling problem as previously discussed.

### SUMMARY OF THE INVENTION

The purpose and advantages of the invention will be set forth in or otherwise apparent from the description and drawings that follow, as well as will be learned by practice of the invention.

The present invention generally relates to therapeutic devices having complementary fastening portions such as Velcro-type hook-and-loop fastener fabrics. More particularly, the invention relates to such therapeutic devices having such fastening portions for adjustably applying a therapeutic device to a part of the body, such as the torso or a limb.

A therapeutic device consistent with the present invention provides at least one strap having a first fastening portion on a first surface and a first complementary fastening portion of substantially similar or greater area on the device for removably attaching the strap to the device to effect adjustment or application thereof around the body part. The straps may comprise essentially the entirety of the device. Alternatively, they may be integrally connected to a central region for wrapping partly around a body or limb, or be attached to a central region of a therapeutic device.

The therapeutic device of the present invention provides an auxiliary strap holding position for the at least one strap, such that the strap can be temporarily attached to the auxiliary strap holding position on the device to prevent tangling when the strap is not in a position used to attach the device to the body, for example while the device is being donned, doffed, or stored. The advantages of the invention are most apparent when a plurality of flexible straps are provided in close proximity to each other.

In a first aspect of the invention, a second complementary fastening portion comprises an auxiliary strap holding position which is located on a surface other than the first complementary fastening portion that is used for removably attaching the device to a part of the body.

In another aspect of the invention, the second complementary fastening portion comprising the auxiliary strap holding position has a surface which is smaller than the surface of the first complementary fastening portion.

In yet another aspect of the invention, the device provides an auxiliary strap holding position for the at least one strap, wherein the second complementary fastening portion that comprises an auxiliary strap holding position has a surface that is smaller than the surface of the first fastening portion on the first surface of the strap.

In a further aspect of the invention, a therapeutic device provides an auxiliary strap holding position for the at least one strap, wherein the surface of the auxiliary strap holding position has a second complementary fastening portion that is smaller than the surface of the first fastening portion on the first surface of the strap, and wherein the auxiliary holding position is located on the opposite side of the strap relative to the first complementary fastening portion.

In a further aspect of the invention, the second complementary fastening portion comprising the auxiliary strap holding position may be positioned such that it is surrounded by, adjacent to, or near to a surface that is not constructed to be a complementary attachment portion. In such an embodiment, the connecting properties of the strap are effectively “disarmed” by holding at least part of the first fabric fastening portion against a surface that does not have a complementary fabric attachment portion, thereby reducing the holding power while also preventing the strap from tangling and attaching to the device elsewhere. Such a device may have a hook fabric or component portion of a hook-and-loop fastener system proximate one end of a strap, with a complimentary loop fabric or portion of substantially similar or greater surface area provided as a primary attachment area for use of the device. In the midst of, adjacent to, or near to a surface without loop fabric, such as on the opposite side of the strap, a small patch or area of loop fabric is provided such that at least a part of the strap having the hook portion may be positioned substantially over the area not having loop fabric but may still be retained on the small loop fabric portion, thereby rendering ineffective part of the area of the hook portion and holding the strap in an auxiliary strap holding position until the strap is released and repositioned to the primary use position.

In still a further aspect of the invention, a complementary or second fabric fastening portion comprising the auxiliary strap holding position may be positioned such that it is surrounded by, adjacent to, or near a first fabric fastening portion. The connecting properties of the strap are “disarmed” by holding at least part of the first fabric fastening portion face-to-face with itself, thereby reducing the holding power and preventing the strap from tangling and attaching to the device elsewhere. The device may have the hook component portion of a hook-and-loop fastener system proximate one end of a strap, with a complimentary loop nap portion of substantially similar or greater size provided on a target or primary use attachment area of the device. In the midst of, adjacent to, or near to the portion having the hook surface, a small portion of loop fabric is provided such that at least a portion of the hook fabric is positioned substantially face-to-face with itself, thereby rendering ineffective part of the hook portion and holding the strap in an auxiliary strap holding position until it is released and repositioned to the primary use position.

In a further aspect of the invention, a method is presented for donning a therapeutic device where at least one strap is secured in an auxiliary strap holding position before donning, preventing tangling of the strap while it is not being used to attach the device to a body, for example, before it is placed in an intended adjustment or releasable attachment position.

In another aspect of the invention, a method is presented for donning a therapeutic device wherein at least one strap end is secured in an auxiliary strap holding position before donning, so as to retain the strap in a position while not being used to attach the device to a limb and to hold the strap end in a position for convenient retrieval of the strap end to thereafter attach the strap end in an intended adjustable and releasable attachment position during donning of the device.

In still a further aspect of the invention, a method is presented for doffing a therapeutic device where at least one strap end is secured in an auxiliary strap holding position while doffing, so as to prevent tangling after having been removed from an intended adjustable and releasable attachment position.

In a further aspect of the invention, a method is presented for doffing a therapeutic garment where at least one strap end is secured in an auxiliary strap holding position while doffing, so as to retain the strap in a position while not being used to attach the device to a limb and to hold the strap end in a position for convenient retrieval and placement of the strap end to an intended adjustable and releasable attachment position during donning of the device.

The therapeutic device of the present invention provides a significant advance over known therapeutic devices in that the connecting properties of the straps can be temporarily “disarmed” while donning, doffing, or storing the device. The therapeutic device of the present invention also provides an auxiliary strap holding position for a strap where the effort to remove the strap from the auxiliary strap holding position and to restore its connective properties is reduced relative to the effort required to remove the strap from its normal connective or primary operating adjustment position.

The therapeutic device of the present invention also provides auxiliary strap holding positions where straps can be temporarily retained in locations that are convenient for retrieval when they later need to be returned to their primary adjustable and releasable attachment positions. As a result, the ease of using therapeutic devices having the auxiliary strap holding positions is greatly enhanced, and particularly so for users having reduced mobility or strength. The advantages of the invention are most apparent when a plurality of straps are provided in close proximity to each other.

It is to be understood that both the foregoing general description and the following detailed description are exemplary and provided for the purposes of explanation only, and are not restrictive of the invention, as claimed. Further features and objects of the present invention will become more fully apparent in the following description of the preferred embodiments and from the appended claims.

### BRIEF DESCRIPTION OF THE DRAWINGS

In describing the preferred embodiments, reference is made to the accompanying drawing figures wherein like parts have like reference numerals, and wherein:

FIG. 1 is a front view of a first embodiment of a therapeutic device consistent with the present invention and shown with the straps laid open and flat.

FIG. 2 is a partial perspective view of the first embodiment shown with greater detail for strap and device construction, and showing a strap in an auxiliary strap holding position.

FIG. 3 is a partial perspective view of a portion of the first embodiment shown with a strap in a primary adjustable and releasable attachment position.

FIG. 4 is a partial perspective view of a first alternative strap construction to that shown in FIGS. 1-3, with a strap in an auxiliary strap holding position.

FIG. 5 is a partial perspective view of a second alternative strap construction to that shown in FIGS. 1-3, with a strap connected to and extending from the inner surface of a band which has an auxiliary strap holding position.

FIG. 6 is a partial perspective view of a third alternative strap construction to that shown in FIGS. 1-3, with a strap connected to and extending from the outer surface of a band, and where the strap has an auxiliary strap holding position.

FIG. 6A is a further partial perspective view of the third alternative strap construction shown in FIG. 6, but with the strap folded over on itself into an auxiliary strap holding position.

FIG. 7 is a partial perspective view of a fourth alternative strap construction shown in FIGS. 1-3, with an auxiliary strap holding position and with a the strap laid open and flat.

FIG. 8 is a partial perspective view of a second preferred embodiment shown with a band having a loop surface on both sides except for a hook portion on the inner surface of a strap at the left end and a non-attachment portion on the band that defines an auxiliary strap holding position.

FIG. 9 is a front view of a third preferred embodiment of a therapeutic device of an alternative construction consistent with the present invention and shown with the device laid open and flat.

FIG. 9A is a partial perspective view of a first alternative strap construction to that shown in FIG. 9, with a strap formed by the band extending beyond the distal end of an attachment portion, and an auxiliary strap holding position formed on the strap.

FIG. 10 is a perspective view of a fourth preferred embodiment of a therapeutic device of an alternative construction consistent with the present invention and shown with one strap laid open and flat and a second strap folded over on itself into an auxiliary strap holding position.

FIG. 11 is a perspective view of a fifth preferred embodiment of a therapeutic device of an alternative construction consistent with the present invention and shown with a pair of straps having auxiliary strap holding positions but with the straps in their primary attachment position.

FIG. 12 is a perspective view of a sixth preferred embodiment of a therapeutic device of an alternative construction consistent with the present invention and shown with an elastic band having an attachment portion and an auxiliary strap holding position at each end.

It should be understood that the drawings are not to scale. While some mechanical details of a therapeutic device, including other plan and section views of the particular components, have been omitted, such details are considered well within the comprehension of those skilled in the art in light of the present disclosure. It also should be understood that the present invention is not limited to the preferred embodiments illustrated.

### DETAILED DESCRIPTION OF THE PREFERRED EMBODIMENTS

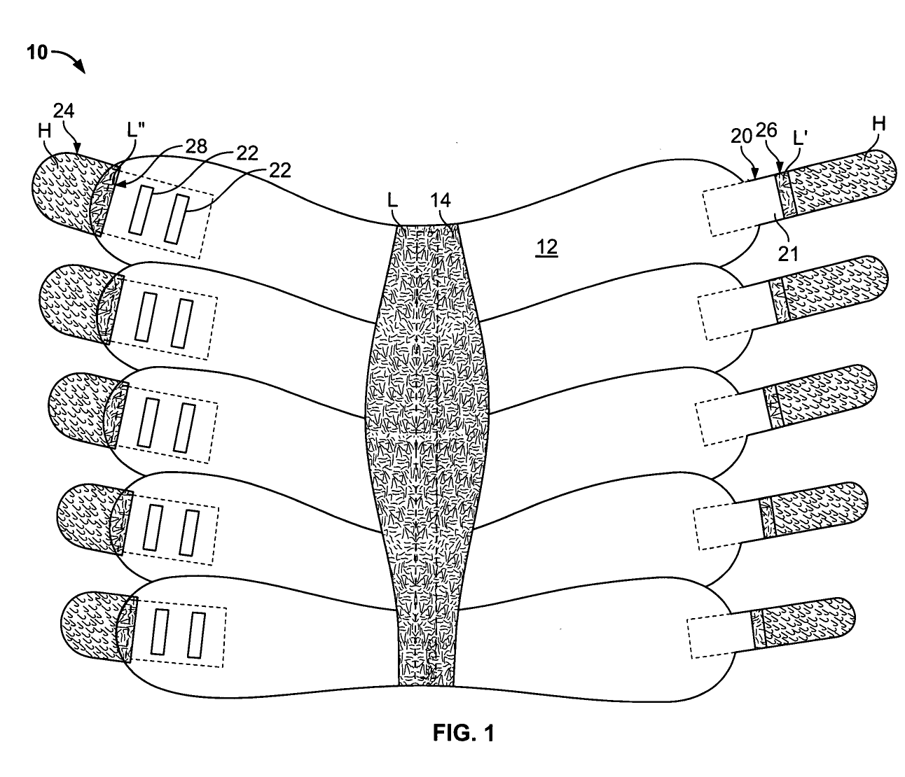

Referring generally to FIGS. 1-12, it will be appreciated that therapeutic devices having auxiliary strap holding positions and being consistent with the present invention may be embodied within numerous configurations.

Referring to a first preferred embodiment shown in FIGS. 1-3, a therapeutic device 10, embodying the present invention is constructed as a compression garment. Device 10 has a plurality of compression bands 12 arranged in a parallel overlapping relationship to enable the device to encompass a body part or limb, such as a leg. In this example, compression bands 12 are held in place with respect to each other by a longitudinal band 14 of fabric sewn to a central region of each compression band 12. Longitudinal band 14 has an inner surface having a loop fabric. In the preferred embodiment, a flexible support 16, such as a strip of semi-rigid plastic, is sewn across the outside of compression bands 12, and a further longitudinal band 18 comprising fabric having an outer loop surface overlays flexible support 16 and is sewn to compression bands 12. In the preferred embodiment, second longitudinal band 18 is positioned in alignment with first longitudinal band 14 on the opposite side of compression bands 12.

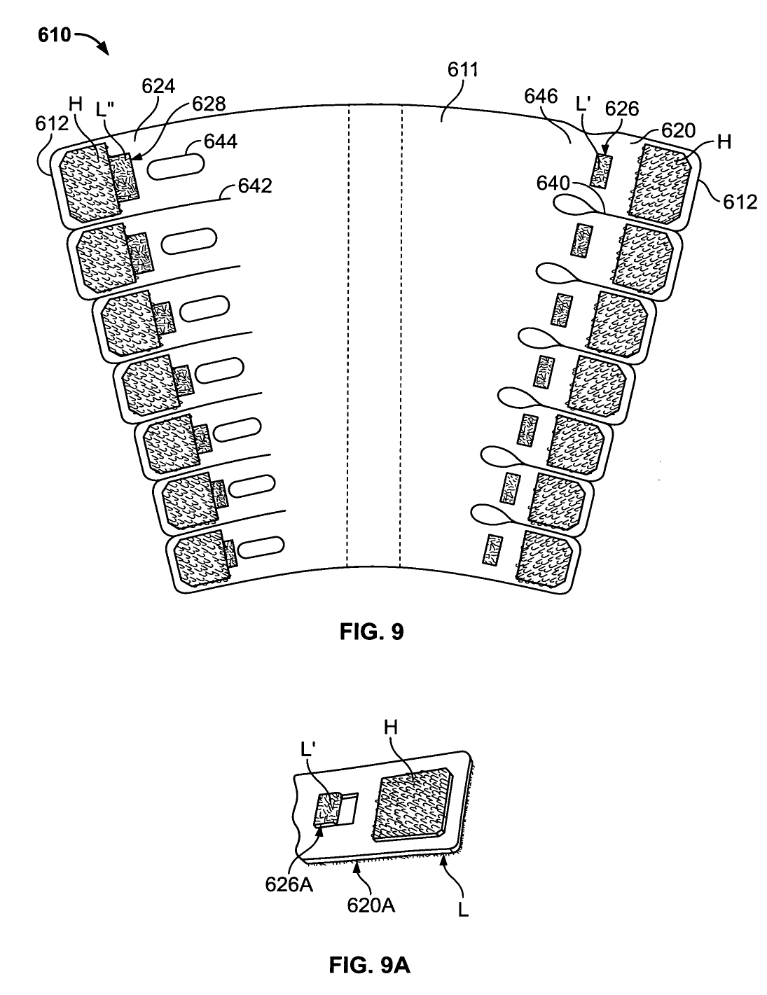

Each compression band 12 has a first strap 20 at a first end. In this example, strap 20 is made from a portion 21 of material extending from band 12, and which is folded back on itself to present inward facing portion L′. Strap 20 also includes an inward facing hook portion H affixed to and extending from the outer surface of portion 21. Each band 12 also has at least one slot 22 near a second end to accommodate first strap 20 to pass through the slot in threaded relationship so as to allow a user to apply compression to the body part encompassed by the device. It will be appreciated that additional slots 22 may be provided to permit a wider range of adjustment in each compression band 12. A second strap 24 is provided at a second end of each band 12. Each first strap 20 and second strap 24 may extend from a respective end of band 12, as shown in FIGS. 1-3, or may overlap or be within the area of band 12, so that the band extends to the end of each strap or beyond, as shown for instance in the fourth preferred embodiment in FIGS. 9 and 9A.

In the embodiment of FIGS. 1-3, each compression band 12 preferably is made from a flexible, foldable Velcro-type fabric having an outer surface loop-portion L which is preferably a light weight loop fabric that is essentially inelastic, although it will be understood that it may comprise elastic material if desired, and may cover an area which is less than the entire outer surface. Each strap comprises an inner surface hook portion H having a strip of Velcro-type hook fabric or tape connected to or extending from the end of a band 12 or as part of a strap. It will be appreciated that each hook portion H may be connected to band 12 or a strap by stitching, adhesive or any other suitable joining methods and materials.

In use, when strap 20 is threaded through a slot 22, the ends of straps 20 and 24 may be pulled away from each other to tighten the band, as shown in FIG. 3. Once a band 12 has been tightened, it can be held in a tightened primary adjustment or releasable holding position by pressing hook portions H of first strap 20 and second strap 24 against the underlying complementary loop portions L on the outer surface of band 12. In the tightened position, first strap 20 is accommodated through a slot 22.

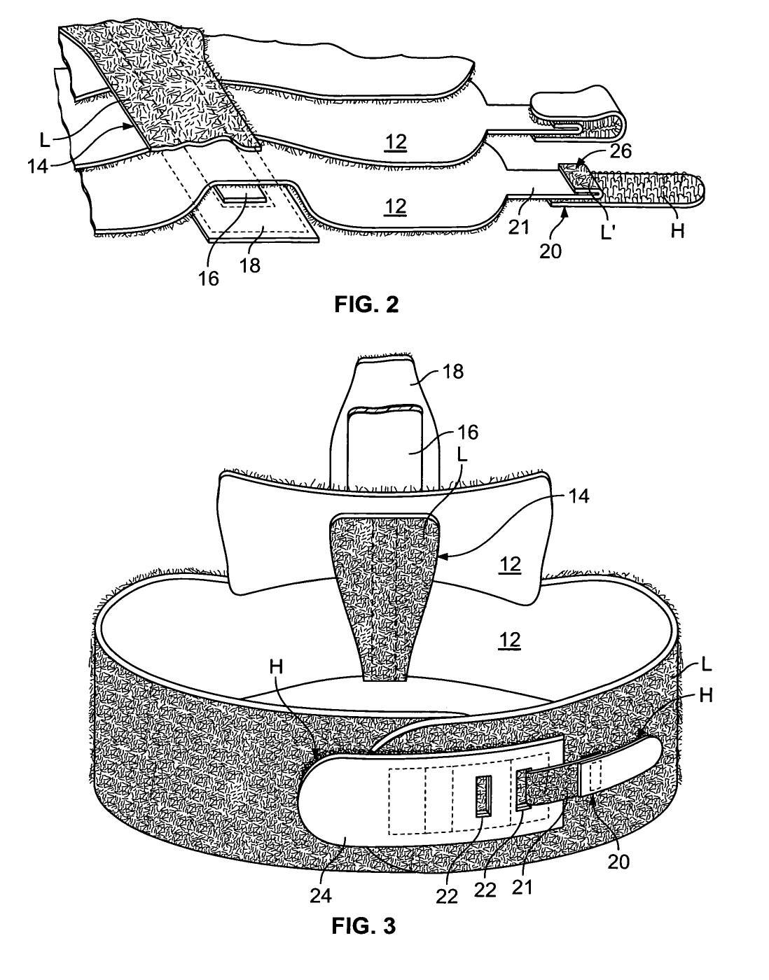

In this first embodiment, a small complimentary loop portion L′ comprising an auxiliary strap holding position 26 is provided near the edge of each hook portion H opposite the distal end of each strap 20, 24. When straps 20, 24 are not being used to hold the compression band in a tightened position, the end of each strap may be folded over on itself with part of a hook portion H pressed into small loop portion L′ of auxiliary strap holding position 26, as shown in FIG. 2, to engage the auxiliary strap holding position. In this position, hook portion H is shielded such that strap 20 will not make firm engagement with any other loop portion L on any of bands 12, or on any other portion of device 10, or on the user's clothing, such as socks (not shown). Thus, strap 20 is prevented from tangling. In addition, with loop portion L′ comprising an auxiliary strap holding position 26 being smaller than hook portion H of strap 20, the force required to separate strap 20 from auxiliary holding position 26 will be less than the force required to remove strap 20 from the primary holding position on the outer surface loop portion L of respective band 12, because the primary holding position offers sufficient area to engage substantially the entirety of hook portion H on strap 20. A strap 24 at the opposite end of each band 12 presents a similar small loop portion L″ comprising an auxiliary strap holding position 28, to be used in the same manner.

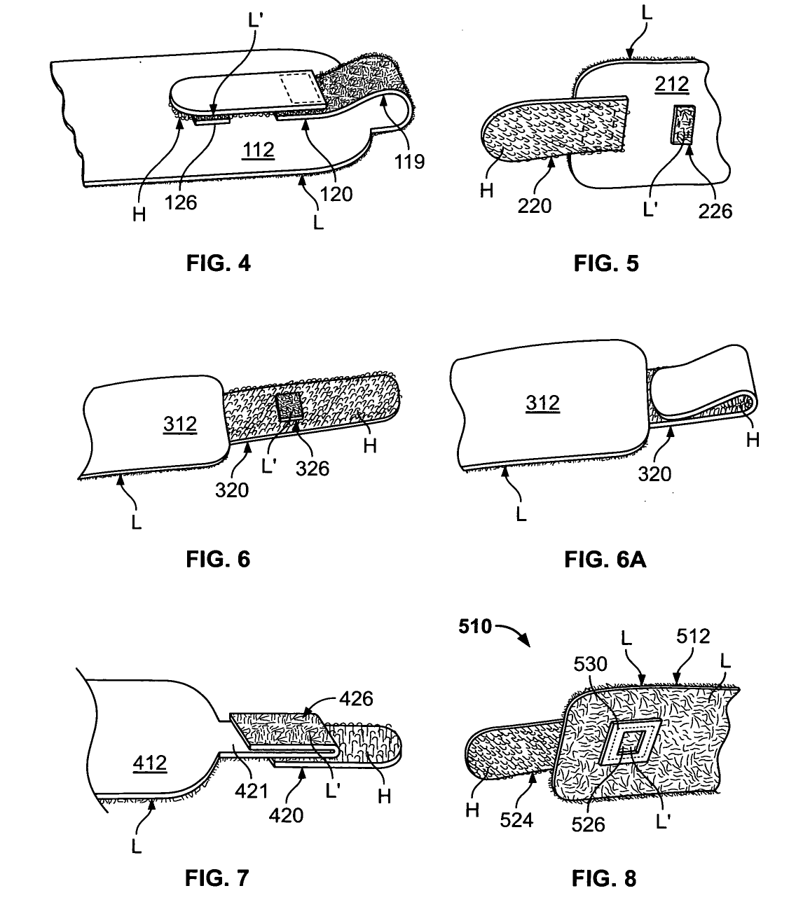

In a first alternative strap construction for the first preferred embodiment, as shown in FIG. 4, a strap 120 extends from band 112. Strap 120 is made from a portion 119 of material extending from band 112, and further includes an inward facing hook portion H which is affixed to and extends from the outer surface of portion 119. As with all of the various constructions of the different materials intended to be affixed to each other within the present device, hook portion H may be affixed to portion 119 by any conventional connecting methods and materials, as previously discussed.

A complementary smaller loop portion L′ is positioned within and connected to a region of band 112, which otherwise does not present hook or loop elements. The small loop portion L′ forms an auxiliary strap holding position 126. When a strap 120 is not being used to hold compression band 112 in a tightened primary holding position, the end of strap 120 may be folded over on itself and part of hook portion H may be pressed into loop portion L′ on auxiliary strap holding position 126. In this position, hook portion H is shielded from making firm engagement with any other portion of loop surface on any further bands 112, or on any other portion of the device, or on the user's clothing. Also, since part of hook portion H on strap 120 overlays an area void of loop fabric, the force required to separate the strap 120 from auxiliary strap holding position 126 will be less than the force required to remove strap 120 from a primary holding position which would offer sufficient area to completely engage the entirety of hook portion H.

In a second alternative strap construction for the first preferred embodiment, as shown in FIG. 5, a strap 220 is affixed to the inner surface of band 212, and comprises an inward facing hook portion H. Similarly to the alternative shown in FIG. 4, a complementary smaller loop portion L′ is positioned within and connected to a region of band 212, which otherwise does not present hook or loop elements, to form an auxiliary strap holding position 226. This alternative construction provides a similar ability for a user to fold over on itself and releasably connect a hook portion H of a strap 220 to an auxiliary strap holding position 226, when not in a tightened primary holding position. As with the prior example, in this position, hook portion H is shielded from making firm engagement with any other loop surface on the device or on the user's clothing. Auxiliary strap holding position 226 also requires reduced effort to separate strap 220 from auxiliary strap holding position 226 because part of hook portion H on strap 220 overlays an area void of loop fabric.

In a third alternative strap construction for the first preferred embodiment, as shown in FIGS. 6 and 6A, a strap 320 having an inward facing hook portion H for releasable attachment to an outer surface loop portion L of a band 312 is affixed to the outer surface of band 312. Strap 320 also has a small loop portion L′ which is complementary to hook portion H, and is positioned within and connected to hook portion H, thereby forming an auxiliary strap holding position 326, as shown in FIG. 6. When a plurality of straps 320 are not being used to hold a therapeutic device in a tightened primary holding position, each strap 320 may be folded over on itself with part of a hook portion H pressed into a corresponding loop portion L′ to releasably hold the strap 320 on auxiliary strap holding position 326, as shown in FIG. 6A. As with the prior alternative constructions, this preferably shields hook portion H such that strap 320 will not engage any other part of loop portion L on any other portion of the device, or on the user's clothing, preventing tangling. As an added benefit, by having hook portion H on strap 320 overlay a portion of itself, the force required to separate hook portion H from loop portion L′ of auxiliary holding position 326 will be lower than the force required to remove strap 320 from a primary holding position which would otherwise offer sufficient area to engage substantially the entirety of hook portion H on strap 320.

FIG. 7 presents a fourth alternative strap construction for the first preferred embodiment of FIGS. 1-3. Here, band 412 extends at a first end via portion 421 into a strap 420, having an inward facing hook portion H affixed to its outer surface, and having portion 421 folded back on itself to present inward facing portion L′, defining an auxiliary strap holding position 426.

When band 412 is used to encircle a body part, hook portion H of strap 420 may be fully engaged with loop portion L on the outer surface of band 412. When donning, doffing or storing the therapeutic device, strap 420 may be folded over on itself to releasably connect the hook portion H to small loop portion L′ in the auxiliary strap holding position 426. This tends to curl band 412 in a manner that discourages hook portion H on strap 420 from engaging loop portion L on the outward facing side of band 412, and prevents inadvertent tangling of strap 420.

A second preferred embodiment is shown in FIG. 8. The embodiment of FIG. 8 is shown in a simplified format as a compression band 510 with the right end portion of a band 512 removed for convenience of viewing. As shown, band 512 is made of a material having both inner and outer surfaces presenting a loop portion L. Band 512 extends at a first end to form a strap (not shown) also having loop material on its outer surface. Band 512 has a strap 524 at its second end which includes a hook portion H affixed to and extending from the second end of band 512. To provide an auxiliary strap holding position 526, a patch of non-loop material 530, having an open center, is affixed to band 512 proximate the end of band 512 from which strap 524 extends. The open center of patch 530 defines a small loop portion L′ which forms auxiliary strap holding position 526.

Thus, this second preferred embodiment permits band 512 to be wrapped around a body part and hook portion H on strap 524 may be pressed into to loop portion L on the outer surface of band 512 to hold therapeutic device 510 in place. When donning, doffing or storing therapeutic device 510, strap 524 may be folded over on itself so as to press hook portion H into loop portion L′ at auxiliary strap holding position 526. It will be appreciated that the reduced surface area for binding which is presented when engaging hook portion H with loop portion L′ permits easy removal and repositioning of strap 524. It also will be appreciated that this form of shielding or interrupting the binding surface area could be provided by placing on loop portion L a small patch of non-loop material, having virtually any outline and not necessarily having an open center, because while it would shield some of the surface of hook portion H, it would still permit hook portion H to bind to loop portion L where hook portion H does not overlie patch 530. In this manner, fancifully shaped patches 530 could be used as targets to define auxiliary strap holding positions for placement of hook portions H.

A third preferred embodiment is shown as a therapeutic device 610 in FIG. 9. Therapeutic device 610 is shown in a configuration as a compression garment made in one piece from a flexible, foldable Velcro-type fabric having an outer surface loop portion L (not seen in the front view) which is preferably a light weight loop fabric, such as of the type designated as Velcro 3610 or Velcro 3800, the former being substantially inelastic and the latter having a limited stretch at least in the vertical or longitudinal direction.

The therapeutic device 610 of FIG. 9 includes a central region 611 which is wrapped around the body part and a plurality of bands 612 integrally connected to the central region and extending outwardly in opposite directions from both sides of the central region to encompass the body part. The bands 612 have first straps 620 at first ends which are defined by slits 640, and second straps 624 at second ends which are defined by slits 642. Each band 612 has a slot 644 proximate strap 624 to accommodate the opposite strap 620 in threaded, folded relationship to apply compression to the body part encompassed by the garment. The straps 620 which are threaded through slots 644 may include portions 646 of reduced width formed by widening slits 642 separating bands 612, but such narrow width portions are not essential because of the flexible, foldable characteristics of the fabric. The inner surfaces of straps 620, 624 have Velcro-type hook portions H at or near their ends. The opposite straps 620, 624 of each band 612 are extended toward each other and one strap 620 of each band 612 is threaded through slot 644 in the complimentary strap 624, and then tightened to apply the desired compression to the body part. Inner surface hook portions H are then pressed against the outer surface loop portions L of the fabric to anchor the straps in a primary tightened position. The garment is removed by separation of hook portions H from loop portions L of the garment and then unthreading straps 620 from straps 624.

A small loop portion L′ comprising an auxiliary strap holding position 626 is provided on each strap 620, while a small loop portion L″ comprising an auxiliary strap holding position 628 is provided on each strap 624, as shown in FIG. 9 near hook portions H, and opposite the distal end of each strap. When the straps are not being used to hold the compression bands 612 in the tightened position, the end of each band may be folded over itself with a part of each hook portion H pressed into a respective loop portion L′, L″ of auxiliary strap holding position 626, 628, respectively. In this position, each hook portion H is shielded such that the strap will not make firm engagement with any other part of outer surface loop portions L on any of bands 612, central region 611, any other portion of garment 610, or the user's clothing. If the small loop portion L′, L″ comprising an auxiliary strap holding position 626, 628 is smaller than its complimentary hook portion H of strap 620, 624, the force required to separate the strap from the auxiliary strap holding position will be less than the force required to remove the strap from the primary holding position, since the primary holding position offers sufficient area to completely engage the entirety of the hook portion H on each strap.

It will be apparent to one skilled in the art that various configurations or constructions of the present invention, such as for example those described with respect to the first preferred embodiment, may be practiced with the therapeutic device 610 shown in FIG. 9. As an example, one alternative construction for strap 620 of the third preferred embodiment is shown in FIG. 9A. In this alternative construction, a strap 620A includes a hook portion H. Also, an auxiliary strap holding position is formed by cutting a flap 626A in the strap and folding the flap inward, so as to make a small piece of what was the outer loop portion L become an inward facing loop portion L′. It will be appreciated that the opening and flap 626A may be secured such as by stitching, adhesive or other suitable means.

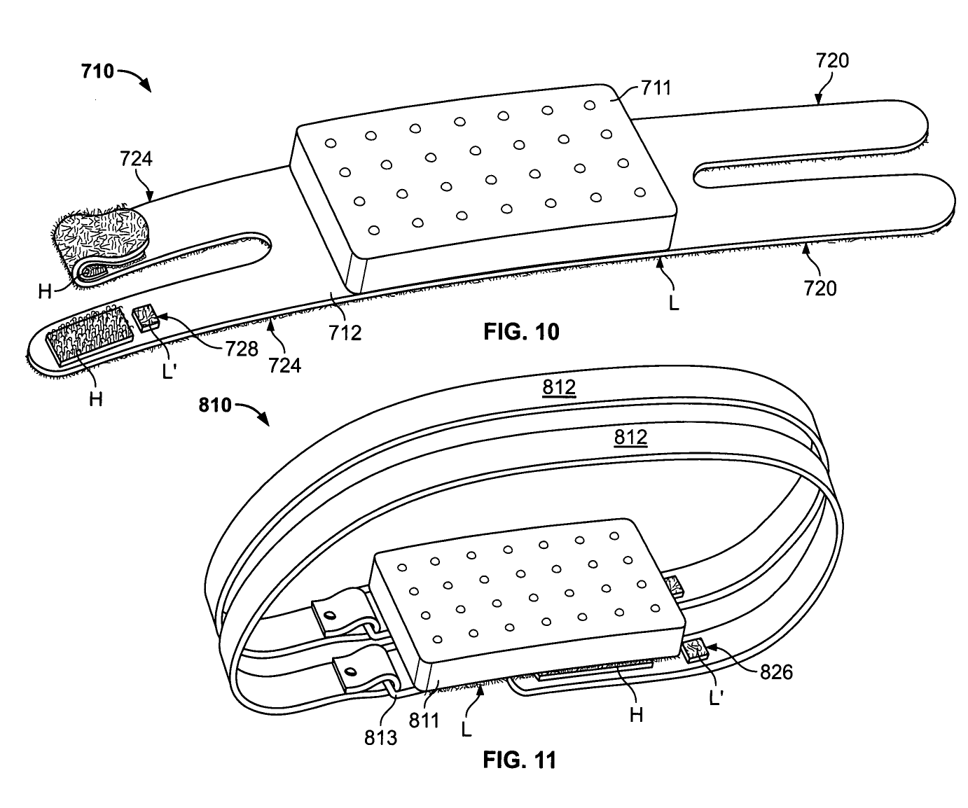

The advantages of the present invention similarly may be realized when practiced with other therapeutic devices worn on the body, such as is shown in FIG. 10. In FIG. 10, a fourth preferred embodiment of the present invention is shown in the form of a therapeutic device 710 that includes a therapeutic pad 711, shown in a simplified manner. It will be appreciated that therapeutic pad 711 represents a variety of potential therapeutic components that may provide a stand alone feature, such as a battery operated light therapy device or a thermal pack, or may include therapeutic components which require power supplied via an external power source, such as may be conveyed by a cord (not shown).

Therapeutic device 710 has a band 712 attached thereto, to permit the device to be worn on a body part, such as around a waist, or in a shorter format, such as around an arm or leg. Band 712 is formed at each end to provide a pair of parallel straps 720 at a first end and 724 at an opposite second end. The entire outer surface of band 712, including straps 720, 724 is a loop portion L of a Velcro-type loop fabric. The inward facing surface of straps 720 have neither a hook nor loop surface. The inward surface of straps 724 are constructed much like straps 620 in FIG. 9, and have a hook portion H and a separate but closely placed small loop portion L′ forming an auxiliary strap holding position 728. Hook portion H and loop portion L′ may be affixed to strap 724 such as by attachment means previously described.

In use, hook portion H on each strap 724 may be connected directly to loop portion L on the outer surface of a strap 720 or band 712, in a primary holding position. Alternatively, each hook portion H may be conveniently prevented from tangling during donning, doffing or storage, by folding each strap 724 over on itself to connect hook portion H to loop portion L′ in the auxiliary strap holding position 728, as shown. As an added benefit, with this embodiment, the force required to separate hook portion H from loop portion L′ will be reduced relative to that required to separate hook portion H from the larger surface of loop portion L.

A fifth preferred embodiment is shown in FIG. 11, with a pad 811 and a pair of straps 812 connected to pad 811 to permit the therapeutic device 810 to be worn around a body part. It will be appreciated that straps 812 may be constructed of various materials, such as nylon, leather, elastic or inelastic fabric, or other suitable materials. In this embodiment, pad 811 has a loop portion L on its outer surface and a pair of rings 813 attached at a first end. Each strap 812 is connected to pad 811 by looping around a ring 813 at the first end, and has an inward facing hook portion H at a second end for releasable connection to loop portion L. To prevent entanglement or inadvertent attachment of hook portion H to loop portion L or to a user's clothing, each strap 812 also has a small inward facing loop portion L′ located relatively close to hook portion H. Thus, when in a use position as shown in FIG. 11, hook portions H are releasably connected to loop portion L, and when not secured around a body part, each hook portion H may be connected to a convenient and complimentary loop portion L′ in an auxiliary strap holding position 826. In this position, hook portion H is shielded and requires less force to be disengaged from loop portion L′ than would be required when removing hook portion H from a primary holding position.

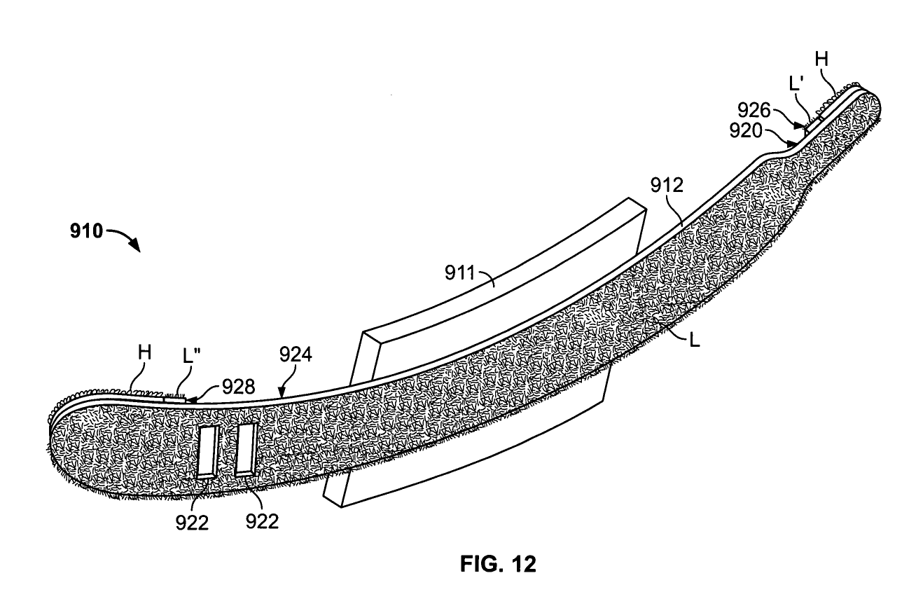

In a sixth embodiment, a therapeutic device 910 is shown in FIG. 12, with a therapeutic component 911 connected to a band 912. Band 912 preferably is made from a flexible, foldable Velcro-type fabric having an outer loop portion L which is preferably a light weight loop fabric that is elastic, but it may be inelastic, if desired. Band 912 has an outer surface loop portion L and is formed with a narrowed strap 920 at a first end, and a full width further strap 924 at a second end. Strap 920 has an inward facing hook portion H proximate its end and a small loop portion L′ adjacent hook portion H. Strap 924 includes a pair of slots 922 to accommodate strap 920 to pass through in threaded relationship. When strap 920 is threaded through a slot 922, the ends of straps 920, 924 are pulled away from each other to tighten the band. Once the band has been tightened, the band can be held in the tightened position to locate therapeutic device 910 on a body part requiring therapy, such as by pressing hook portions H against underlying complementary loop portion L. Additional slots 922 may be provided to permit a wider range of adjustment of band 912.

Small loop portion L′ forms an auxiliary strap holding position 926 on strap 920 and is provided near the edge of hook portion H. A similar small loop portion L″ forms an auxiliary strap holding position 928 at the second end, where it is located near the other hook portion H. When therapeutic device 910 is not being used, the end of strap 920, 924 may be folded over on itself to press part of a hook portion H into a respective loop portion L′, L″ in auxiliary strap holding positions 926, 928. As with the prior embodiments, this provides more convenient use, without tangling by shielding hook portions H from full engagement with loop portion L, any other similar band 912, or the user's clothing.

It will be appreciated that a therapeutic device in accordance with the present invention may be provided in various configurations having complimentary hook and loop portions for releasable engagement, but still provide for an auxiliary strap holding position for each respective strap. Any variety of suitable materials of construction, configurations, shapes and sizes for the components and methods of connecting the components may be utilized to meet the particular needs and requirements of an end user. It will be apparent to those skilled in the art that various modifications can be made in the design and construction of such a therapeutic

What is claimed is:

1. A therapeutic device comprising:
at least one strap having a first fastening portion having a first surface area;
a first complementary fastening portion which is complementary to the first fastening portion of the strap and wherein the first complementary fastening portion has a substantially similar or greater surface area than that of the first fastening portion to permit removable attachment of the device to a body part of a user by encircling the body part with the device, and
a second complementary fastening portion which is complementary to the first fastening portion of the strap and wherein the second complementary fastening portion provides an auxiliary strap holding position.

2. The therapeutic device in accordance with claim 1, wherein the second complementary fastening portion is located on a surface other than on the first complementary fastening portion.

3. The therapeutic device in accordance with claim 1, wherein the first complementary fastening portion has a surface area and the second complementary fastening portion has a surface area that is smaller than the surface area of the first complementary fastening portion.

4. The therapeutic device in accordance with claim 1, wherein the second complementary fastening portion has a surface area that is smaller than the surface area of the first fastening portion.

5. The therapeutic device in accordance with claim 1, wherein the first fastening portion and the first and second complementary fastening portions comprise hook or loop fastening portions.

6. The therapeutic device in accordance with claim 1, wherein the device further comprises a plurality of said straps.

7. A therapeutic device comprising at least one strap having at least one primary holding position for releasable connection of the strap when encircling a body part, and having at least one auxiliary strap holding position for releasable connection of the strap when not encircling the body part.

8. The therapeutic device in accordance with claim 7, wherein the strap can be temporarily connected to the auxiliary holding position on the device to prevent tangling of the strap or inadvertent connection of the strap to other surfaces.

9. The therapeutic device in accordance with claim 7, wherein less force is required to remove the strap from the auxiliary strap holding position than to remove the strap from the primary holding position.

10. A therapeutic device comprising:
at least one strap having a first fastening portion;
a complementary fastening portion located on the device and providing a primary holding position when the device is encircling a body part and the first fastening portion is connected to the complementary fastening portion; and
an auxiliary strap holding position for the at least one strap, wherein the auxiliary strap holding position also has a complementary fastening portion.

11. The therapeutic device in accordance with claim 10, wherein the at least one strap further comprises a second fastening portion;
a second complementary fastening portion located on the device and providing a second primary holding position for the second fastening portion when the device is encircling a body part and the second fastening portion is connected to the second complementary fastening portion; and
a second auxiliary strap holding position for the at least one strap, wherein the second auxiliary strap holding position also has a complementary fastening portion.

12. The therapeutic device in accordance with claim 10, wherein the first fastening portion and complementary fastening portions comprise fabric fastening portions.

13. The therapeutic device in accordance with claim 10, wherein the complementary fastening portion that provides the primary holding position has a surface area and the auxiliary strap holding position has a smaller surface area than that of the primary complementary fastening portion.

14. The therapeutic device in accordance with claim 10, wherein the complementary fastening portion that provides the auxiliary strap holding position has a surface area and the first fastening portion has a surface area and auxiliary strap holding position surface area is smaller than the surface area of the first fastening portion.

15. The therapeutic device in accordance with claim 10, wherein the device further comprises a plurality of said straps and said straps are integrally connected to a central region.

16. A therapeutic device comprising:
at least one strap having a first fastening portion;
a complementary fastening portion located on the device and providing a primary holding position when the device is encircling a body part and the first fastening portion is connected to the complementary fastening portion; and
an auxiliary strap holding position for the at least one strap, wherein the auxiliary strap holding position further comprises a portion which is not constructed of a complementary fastening material and which is located on a complementary fastening portion.

17. The therapeutic device in accordance with claim 16, wherein the portion not constructed of a complementary fastening material is surrounded by, adjacent to, or proximate the complementary fastening portion.

18. The therapeutic device in accordance with claim 16, wherein the first fastening portion and the complementary fastening portion comprise fabric fastening hook or loop material.

19. A method of donning a therapeutic device that has at least one strap, the method comprising: disconnecting the strap from an auxiliary strap holding position, encircling a body part with the device, and moving the strap to a primary holding position thereby maintaining the device in a position encircling the body part.

20. The method of donning a therapeutic device that has at least one strap of claim 19, wherein it requires less force to remove the strap from the auxiliary strap holding position than to remove the strap from the primary holding position.

21. A method of doffing a therapeutic device that has at least one strap having a strap end, the method comprising: removing the strap end from an adjustable and releasable connection position wherein the device is encircling a body part, connecting the strap end to an auxiliary strap holding position wherein the strap end will be prevented from tangling or otherwise becoming inadvertently connected to other surfaces and will be available for convenient retrieval and placement of the strap end to an adjustable and releasable connection position during future donning of the device.

22. The method of doffing a therapeutic device that has at least one strap having a strap end of claim 21, wherein it requires less force to remove the strap end from the auxiliary strap holding position than to remove the strap from the adjustable and releasable connection position. device without departing from the scope or spirit of the present invention, and that the claims are not limited to the preferred embodiments illustrated.
